(19) United States
(12) Patent Application Publication
(10) Pub. No.: US 2024/0226150 A9
(43) Pub. Date: Jul. 11, 2024

(54) PREPARATION METHOD OF CITRULLINATED VIMENTIN ANTIGEN-SPECIFIC IMMUNE TOLEROGENIC DENDRITIC CELLS, AND USES THEREOF

(71) Applicants: THE CATHOLIC UNIVERSITY OF KOREA INDUSTRY-ACADEMIC COOPERATION FOUNDATION, Seoul (KR); SEOUL NATIONAL UNIVERSITY R&DB FOUNDATION, Seoul (KR); Daegu Gyeongbuk Institute of Science and Technology, Dalseong-gun (KR)

(72) Inventors: Kiyuk CHANG, Seoul (KR); Daehee HWANG, Suwon-si (KR); Min-Sik KIM, Suwon-si (KR); Eunmin KIM, Namyangju-si (KR); Eun Hye PARK, Yongin-si (KR); Chan Woo KIM, Suwon-si (KR)

(73) Assignees: THE CATHOLIC UNIVERSITY OF KOREA INDUSTRY-ACADEMIC COOPERATION FOUNDATION, Seoul (KR); SEOUL NATIONAL UNIVERSITY R&DB FOUNDATION, Seoul (KR); Daegu Gyeongbuk Institute of Science and Technology, Dalseong-gun (KR)

(21) Appl. No.: 18/448,665

(22) Filed: Aug. 11, 2023

(30) Foreign Application Priority Data
Aug. 12, 2022 (KR) 10-2022-0101239

(51) Int. Cl. A61K 35/15; A61K 39/00; A61P 9/04; C07K 14/47; C12N 5/00; C12N 5/0784

(52) U.S. Cl. CPC A61K 35/15; A61K 39/4615; A61K 39/4621; A61P 9/04; C07K 14/4702; C12N 5/0018; C12N 5/064

(57) ABSTRACT

The present disclosure relates to the preparation of citrullinated vimentin antigen-specific immune tolerogenic dendritic cells and a composition for preventing or treating heart failure after myocardial infarction comprising the same. According to the present disclosure, it is confirmed that immune tolerogenic dendritic cells differentiated by treating immature dendritic cells with citrullinated vimentin regulate the expression of immune-related factors and have an excellent therapeutic effect on heart failure caused by myocardial infarction.

### TECHNICAL FIELD

The present disclosure relates to a preparation method of citrullinated vimentin antigen-specific immune tolerogenic dendritic cells, and a composition for preventing or treating heart diseases comprising immune tolerogenic dendritic cells prepared through the preparation method.

### BACKGROUND

Recently, although the mortality rate due to acute myocardial infarction has significantly decreased, the number of patients with heart failure due to excessive left ventricular remodeling after infarction is rapidly increasing, and the rapid increase in patients with heart failure after myocardial infarction is emerging as a new health and social issue. After acute myocardial infarction, the left ventricle is remodeled in a process of wound healing of the damaged myocardium, but at this time, when excessive left ventricular remodeling occurs, the left ventricle extends and contractility decreases, resulting in heart failure. To date, no therapy has been developed to suppress the occurrence of heart failure after myocardial infarction, and a therapeutic agent for preventing heart failure after infarction focuses on myocardial regeneration, and cellular therapeutic agents using stem cells have been mainly developed, but cannot effectively prevent heart failure after infarction and thus fall short of expectations.

It has been found that the inflammatory response by immune cells at the infarct site after acute myocardial infarction is the main cause of excessive ventricular remodeling and heart failure, and at this time, Ly-6Chigh as an inflammatory macrophage and Ly-6Clow as a wound repair macrophage have been reported as involved immune cells. At this time, it has been found that the main immune cells that control the inflammatory response in the myocardium after infarction are regulatory T cells (Treg). Immediately after infarction, inflammatory macrophages are activated to cause tissue inflammation, and if this process is delayed, myocardial tissue is excessively destroyed to negatively affect left ventricle remodeling. However, in this process, Treg plays a major role in converting immune cells in tissues from inflammatory macrophages to wound-healing macrophages.

Meanwhile, dendritic cells (DC) are classified into mature DCs (mDCs) and immature DCs (iDCs) according to the degree of differentiation, and unlike the mature DCs, the immature DCs are known to be involved in immune tolerance. Immune tolerogenic dendritic cells promote the induction of differentiation of naive T cells into Treg to exhibit immune tolerance, and in myocardial infarction, Treg cells increase the homing and survival of vascular endothelial progenitor cells (EPCs) into the myocardium, and may significantly improve the recovery of the damaged left ventricle compared to treatment by administration of stem cells after infarction.

Therefore, the present inventors prepared the immune tolerogenic dendritic cells differentiated by a specific method, and confirmed that the immune tolerogenic dendritic cells regulated the expression of immune-related factors and suppressed excessive remodeling of the myocardium and increased the probability of survival when actually injected to a myocardial infarction animal model, thereby improving heart failure after acute myocardial infarction.

### SUMMARY

An object of the present disclosure is to provide a preparation method of immune tolerogenic dendritic cells.

Another object of the present disclosure is to provide a method for inducing differentiation of immature dendritic cells into immune tolerogenic dendritic cells.

Yet another object of the present disclosure is to provide immune tolerogenic dendritic cells.

Yet another object of the present disclosure is to provide a method for preventing or treating heart diseases.

An exemplary embodiment of the present disclosure provides a preparation method of immune tolerogenic dendritic cells including culturing immature dendritic cells in a citrullinated vimentin-containing medium.

Another exemplary embodiment of the present disclosure provides a method for inducing differentiation of immature dendritic cells into immune tolerogenic dendritic cells, including treating immature dendritic cells with citrullinated vimentin.

Yet another exemplary embodiment of the present disclosure provides immune tolerogenic dendritic cells including citrullinated vimentin as an antigen, prepared through the preparation method of the present disclosure.

Yet another exemplary embodiment of the present disclosure provides a method for preventing or treating heart diseases, including administering immune tolerogenic dendritic cells prepared by the preparation method of the present disclosure to a subject.

According to the present disclosure, when immature dendritic cells are treated with citrullinated vimentin to differentiate into immune tolerogenic dendritic cells, it is confirmed that the immune tolerogenic dendritic cells regulate the expression of immune-related factors and may excellently treat heart failure caused by myocardial infarction and thus can be usefully used in related industries.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating an immunopeptidome process of the present disclosure.

FIG. 2 is a diagram illustrating a process of obtaining a therapeutic target antigen of the present disclosure.

FIG. 3 is a diagram illustrating a process of isolating the therapeutic target antigen of the present disclosure into an immunopeptidome.

FIG. 4 is a diagram illustrating a process of confirming target antigen expression according to the induction of myocardial infarction.

FIG. 5 is a diagram analyzing the expression of a target antigen, vimentin according to the induction of myocardial infarction by Western blot.

FIG. 6 is a diagram confirming the mRNA expression of a target antigen according to the induction of myocardial infarction.

FIG. 7 is a diagram illustrating a process of confirming the expression of vimentin in a myocardial infarction area according to the induction of myocardial infarction.

FIG. 8 is a diagram analyzing the vimentin expression according to the induction of myocardial infarction by ELISA.

FIG. 9 is a diagram confirming the expression of vimentin in the myocardial infarction area according to the induction of myocardial infarction by real time PCR. In FIG. 9, **** means p<0.0001.

FIG. 10 is a diagram illustrating a method for verifying a target antigen in vitro in immune tolerogenic dendritic cells according to the present disclosure.

FIG. 11 is a diagram confirming the regulation of expression of immune-related factors in in vitro verification of a target antigen in immune tolerogenic dendritic cells according to the present disclosure.

FIG. 12 is a diagram illustrating a process for preparing immune tolerogenic dendritic cells according to the present disclosure.

FIG. 13 is a diagram confirming the expression of immune-related factors in immune tolerogenic dendritic cells according to the present disclosure by real time PCR. In FIG. 11, ** means p<0.01 and **** means p<0.0001.

FIG. 14 is a diagram illustrating an in vivo verification method for a therapeutic effect of immune tolerogenic dendritic cells according to the present disclosure on heart failure after acute myocardial infarction.

FIG. 15 is a result of confirming a medial thickness (MT) by staining coronary arteries after administering immune tolerogenic dendritic cells according to the present disclosure to an animal model induced with heart failure after acute myocardial infarction.

FIG. 16 is a result of confirming an infarct size after administering immune tolerogenic dendritic cells according to the present disclosure to an animal model induced with heart failure after acute myocardial infarction. In FIG. 14, ** means p<0.01.

FIG. 17 is a result of confirming the probability of survival after administering immune tolerogenic dendritic cells according to the present disclosure to an animal model induced with heart failure after acute myocardial infarction.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, exemplary embodiments of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined that they unnecessarily make the gist of the present disclosure unclear. Terminologies used herein are terminologies used to properly express exemplary embodiments of the present disclosure, which may vary according to a user, an operator's intention, or customs in the art to which the present disclosure pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present disclosure provides a preparation method of immune tolerogenic dendritic cells including culturing immature dendritic cells in a citrullinated vimentin-containing medium.

The vimentin is a cytoskeletal intermediate filament protein present in mesenchymal-origin cells, including leukocytes, endothelial cells and smooth muscle cells. At a high calcium concentration, a citrullinated reaction occurs by peptidyl arginine deiminase 2 (PAD2), which may occur during apoptosis of macrophages. The citrullinated vimentin is known to play a role in the production of anti-citrullinated protein antibody (ACPA). However, as in the present disclosure, a technique for differentiating into immune tolerogenic dendritic cells using citrullinated vimentin has not been disclosed.

In the present disclosure, the citrullinated vimentin may be included in the medium at a concentration of 10 to 1,000 ng/ml, preferably at a concentration of 50 to 300 ng/ml, more preferably at a concentration of 100 ng/ml, but is not limited thereto.

If the citrullinated vimentin is included below the concentration range, differentiation efficiency as desired may not be obtained, and if the citrullinated vimentin is included above the concentration range, a problem of over-differentiation more than desired may be caused.

In addition, in the preparation method of the present disclosure, the medium may further include Troponin I.

According to an exemplary embodiment of the present disclosure, it was confirmed that the expression of immune regulation factors was significantly increased in immune tolerogenic dendritic cells differentiated by treatment with citrullinated vimentin alone or in combination with citrullinated vimentin and Troponin I.

The Troponin I may be included in the medium at a concentration of 0.5 to ng/ml, preferably at a concentration of 0.5 to 5 ng/ml, more preferably at a concentration of 1 ng/ml, but is not limited thereto.

As used herein, the term “immune tolerance” refers to a state in which an immune response to a specific antigen is not exhibited. Therefore, the “immune tolerogenic dendritic cells” refer to dendritic cells that induce tolerance to self-antigens and inhibit the proliferation of T cells, and for the purpose of the present disclosure, the immune tolerogenic dendritic cells of the present disclosure refer to dendritic cells that induce immune tolerance against self-antigens that cause heart disease, preferably heart failure after myocardial infarction.

The immune tolerogenic dendritic cells of the present disclosure are obtained by inducing differentiation of immature dendritic cells. The immature dendritic cells may be directly obtained from bone marrow, spleen, lymph, thymus or blood of animals, or obtained by culturing dendritic cell precursors present in them, for example, pluripotent cells, hematopoietic stem cells, progenitor cells, peripheral blood mononuclear cells, CD14+ monocyte cells or CD34+ monocyte cells in the presence of suitable cytokines. Suitable cytokines used to obtain the immature dendritic cells include granulocyte macrophage colony stimulating factor (GM-CSF), interleukin-4 (IL-4), IL-13, IL-7 and tumor necrosis factor-α (TNF-α), but are not limited thereto. In the cytokines, a mixture of GM-CSF and IL-4, a mixture of GM-CSF and IL-13, a mixture of GM-CSF and IL-7, or a mixture of GM-CSF, IL-4 and TNF-α may be used.

The immature dendritic cells may be obtained from bone marrow, spleen, lymph, thymus or blood, but are not limited thereto.

In addition, the immature dendritic cells may be derived from mammals, and the mammals may include, for example, mice, rats, guinea pigs, hamsters, rabbits, cats, dogs, sheep, pigs, cows, horses, goats, monkeys, and humans.

As the medium, any medium generally used for culturing animal cells may be used. Preferably, a medium containing serum (e.g., fetal bovine serum, horse serum or human serum) may be used. As used herein, the medium includes, for example, RPMI series (e.g., RPMI 1640), Eagle's minimum essential medium (Eagle's MEM, Eagle), α-MEM, Iscove's MEM, 199 medium, CMRL 1066, F12, F10, Dulbecco's modification of Eagle's medium (DMEM), a mixture of DMEM and F12, Way-mouth's MB752/1, McCoy's 5A and MCDB series, but is not limited thereto. The medium may contain other components, such as antioxidants (e.g., β-mercaptoethanol) or antibiotics (e.g., penicillin/streptomycin).

In an exemplary embodiment of the present disclosure, in the medium containing the citrullinated vimentin alone or both citrullinated vimentin and Troponin I, GM-CSF, IL-4 and TNF-α are further added.

Accordingly, in the preparation method of the present disclosure, the medium may further include at least one selected from the group consisting of a granulocyte macrophage colony stimulating factor (GM-CSF), interleukin-4 (IL-4) and tumor necrosis factor-α (TNF-α).

In the present disclosure, the culture may be performed for 2 hours to 24 hours. The culture may be performed preferably for 2 hours to 12 hours, more preferably for 2 hours to 6 hours, and much more preferably for 4 hours.

In the present disclosure, the immature dendritic cells are treated with the citrullinated vimentin alone or the mixture of citrullinated vimentin and Troponin I simultaneously with GM-CSF, IL-4 and TNF-α to differentiate into immune tolerogenic dendritic cells.

Alternatively, the immature dendritic cells are treated with GM-CSF, IL-4 and TNF-α and then treated with the citrullinated vimentin alone or the mixture of citrullinated vimentin and Troponin I to differentiate into immune tolerogenic dendritic cells.

Further, the present disclosure provides a method for inducing differentiation of immature dendritic cells into immune tolerogenic dendritic cells, including treating immature dendritic cells with citrullinated vimentin.

Through the method, the immature dendritic cells may be differentiated into immune tolerogenic dendritic cells in vitro. Through this, the immune tolerogenic dendritic cells proliferated ex vivo may be transplanted to a lesioned area later to be usefully used for cell therapy.

Through the method, the immature dendritic cells may be differentiated into immune tolerogenic dendritic cells in vivo. Specifically, after the immature dendritic cells are transplanted to the lesion site, the citrullinated vimentin is continuously injected into the transplant site to effectively generate immune tolerogenic dendritic cells in vivo. This is a method of transplanting immature dendritic cells to a lesion site that requires immune tolerogenic dendritic cells before differentiation and differentiating the immature dendritic cells into immune tolerogenic dendritic cells, so that in heart diseases, restoration of heart function may be expected through immature dendritic cell transplantation therapy.

In addition, the present disclosure may provide a reagent composition for inducing differentiation of immature dendritic cells including citrullinated vimentin into immune tolerogenic dendritic cells. In addition, the present disclosure may provide a medium composition for inducing differentiation of immature dendritic cells including citrullinated vimentin into immune tolerogenic dendritic cells.

In the present disclosure, the media mean media capable of supporting cell growth and survival in vitro, and include all conventional media used in the art suitable for cell culture. The medium and culture conditions may be selected according to a type of cell. The medium used for culture is preferably a cell culture minimum medium (CCMM), and generally includes a carbon source, a nitrogen source, and trace elements. Such cell culture minimal media include, for example, Dulbecco's Modified Eagle's Medium (DMEM), Minimal essential Medium (MEM), Basal Medium Eagle (BME), RPMI1640, F-10, Ham's F12, a Minimal essential Medium (αMEM), Glasgow's Minimal essential Medium (GMEM), and Iscove's Modified Dulbecco's Medium (IMDM), but are not limited thereto. In addition, the media may include antibiotics such as penicillin, streptomycin, and gentamicin.

In the present disclosure, when citrullinated vimentin is treated in vitro, immature dendritic cells may be treated during continuous subculture, and if necessary, the immature dendritic cells may be continuously treated while exchanging citrullinated vimentin with the medium.

The term “differentiation” of the present disclosure means a phenomenon in which a relatively simple system is generally divided into two or more qualitatively different partial systems, particularly, a phenomenon in which cells are divided and proliferated to be specified into different structures or functions during growing, that is, a phenomenon in which cells, tissues, and the like of an organism are changed into forms or functions for performing each given work.

The method for measuring the degree of differentiation of immune tolerogenic dendritic cells differentiated by the preparation method of the present disclosure is not particularly limited thereto, but may use methods known in the art, such as a flow cytometry method, an immunocytochemical method, a method for measuring changes in cell surface markers or morphology using PCR or gene-expression profiling, a method for examining cell morphological changes using an optical microscope or confocal microscope, and a method of measuring changes in gene expression profiles. Preferably, RT-PCR, Oil-red O staining, Safranin O staining, Type II collagen immunohistochemical staining, alkaline phosphate (ALP) staining, or Alizarin red S staining may be used.

Further, the present disclosure provides immune tolerogenic dendritic cells including citrullinated vimentin as an antigen, prepared through the preparation method of the present disclosure.

According to an exemplary embodiment of the present disclosure, the immune tolerogenic dendritic cells may further include Troponin I as an antigen.

In addition, according to an exemplary embodiment of the present disclosure, the immune tolerogenic dendritic cells may increase the expression of at least one factor selected from the group consisting of Ido, IL-10, IL-12b and IL-6.

In addition, according to an exemplary embodiment of the present disclosure, the immune tolerogenic dendritic cells may suppress the expression of at least one factor selected from the group consisting of TGF-beta and CCR2.

In particular, according to an exemplary embodiment of the present disclosure, it was confirmed that the immune tolerogenic dendritic cells had the effect of regulating the expression of immune-related factors as described above, and simultaneously had an excellent treating effect in animal models with heart failure caused by myocardial infarction.

Therefore, the present disclosure provides a method for preventing or treating heart diseases including administering to a subject immune tolerogenic dendritic cells prepared by the preparation method according to the present disclosure. In the present disclosure, in an exemplary embodiment, the immune tolerogenic dendritic cells may be provided as a cellular therapeutic agent.

The term “cellular therapeutic agent” used herein refers to a drug (US FDA regulation) used for the purpose of treatment, diagnosis, and prevention by cells and tissues prepared through isolation, culture, and special manipulation from humans. In addition, the cellular therapeutic agent means a drug in which these cells are used for the purpose of treatment, diagnosis, and prevention of diseases through a series of actions, such as proliferating and selecting living autologous, allogeneic, or heterogeneous cells in vitro, or changing the biological characteristics of cells by other methods, to restore the function of cells or tissues.

When the immune tolerogenic dendritic cells of the present disclosure are provided as the cellular therapeutic agent, the cellular therapeutic agent may be administered directly to the heart lesion site, which may be administered in the form of injections. In this case, a hydrogel may be further included to be suitable for injections.

The hydrogel that may be used in the cellular therapeutic agent of the present disclosure may include hydrogels known in the art suitable for injections without limitation, and may be at least one selected from the group consisting of small intestine submucosal tissue, hyaluronic acid, carboxymethylcellulose (CMC), alginate, chitosan, polyacrylamide, poly(N-isopropylacrylamide), β-glycerophosphate, poly(ethylene oxide)poly(propylene oxide)poly(ethyleneoxide) (Pluronic), fibrin, polyethylene oxide (PEO), and a mixture of carboxymethyl cellulose (CMC) and polyethyleneimine (PEI), but is not limited thereto.

A preferable dose of the cellular therapeutic agent composition of the present disclosure varies according to the condition and body weight of a subject, the degree of a disease, a drug form, and the route and period of administration, but may be properly selected by those skilled in the art. The administration may also be performed once a day or performed in several divided doses, and the dosage is not intended to limit the scope of the present disclosure in any way.

Further, in the treatment method of the present disclosure, in an exemplary embodiment, the immune tolerogenic dendritic cells may be provided as a pharmaceutical composition.

The heart disease may be at least one selected from the group consisting of myocardial infarction, myocarditis, heart failure and cardiomyopathy, preferably myocardial infarction or heart failure, more preferably heart failure, and even more preferably heart failure after acute myocardial infarction.

In the present disclosure, the “heart failure after acute myocardial infarction” is heart failure due to excessive left ventricular remodeling after infarction, that is, heart failure occurring when in a process of remodeling the left ventricle in a process of wound healing of the damaged myocardium after acute myocardial infarction, excessive left ventricular remodeling is induced, causing the left ventricle to expand and contractility to decrease.

In the present disclosure, the “subject” means a target subject who has or is likely to develop heart diseases, and the “subject” may mean all animals including humans.

As used herein, the term “prevention” refers to all actions that suppress the symptoms of a specific disease or delay the progression thereof by administering the immune tolerogenic dendritic cells according to the present disclosure and/or a composition containing the same.

As used herein, the term “treatment” refers to all actions that ameliorates or beneficially alters the symptoms of a specific disease by administering the immune tolerogenic dendritic cells according to the present disclosure and/or the composition containing the same.

When the immune tolerogenic dendritic cells according to the present disclosure are provided as a pharmaceutical composition, the pharmaceutical composition may further include an adjuvant in addition to the active ingredient. The adjuvant may be used with any adjuvant known in the art without limitation, but further includes, for example, Freund's complete adjuvant or incomplete adjuvant to increase the effect thereof.

In addition, the pharmaceutical composition may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition according to the present disclosure may be formulated and used in the form of oral formulations such as powders, granules, tablets, capsules, suspensions, emulsions, syrups, and aerosols, external preparations, suppositories, and sterile injectable solutions according to each conventional method.

The formulations may be prepared by using diluents or excipients, such as a filler, an extender, a binder, a wetting agent, a disintegrating agent, and a surfactant, which are generally used. Solid formulations for oral administration include tablets, pills, powders, granules, and capsules, and these solid formulations may be prepared by mixing at least one excipient, for example, starch, calcium carbonate, sucrose, lactose and gelatin with the active ingredient. Further, lubricants such as magnesium stearate and talc may be used in addition to simple excipients. Liquid formulations for oral administration may correspond to suspensions, oral liquids, emulsions, syrups, and the like, and may include various excipients, for example, a wetting agent, a sweetener, an aromatic agent, and a preserving agent, in addition to water and liquid paraffin which are commonly used diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, and injectable ester such as ethyl oleate may be used. As a base of the suppository, witepsol, Tween 61, cacao butter, laurinum, and glycerogelatin may be used.

The immune tolerogenic dendritic cells according to the present disclosure and/or the pharmaceutical composition including the same may be administered to a subject through various routes. All methods of administration may be expected, and the pharmaceutical composition may be administered, for example, oral, intravenous, intramuscular, subcutaneous, and intraperitoneal injection.

The dose of the pharmaceutical composition according to the present disclosure is selected in consideration of the age, body weight, sex, and physical condition of the subject. It is obvious that the concentration of the active ingredient included in the pharmaceutical composition may be variously selected according to a subject, and preferably included in the pharmaceutical composition at a concentration of 0.01 μg/ml to 5,000 μg/ml. When the concentration is less than 0.01 μg/ml, pharmaceutical activity may not be shown, and when the concentration exceeds 5,000 μg/ml, toxicity to the human body may be exhibited.

In addition to the immune tolerogenic dendritic cells as the active ingredient, the pharmaceutical composition according to the present disclosure may further include any compound or natural extract that has already been verified for safety in order to increase the therapeutic effect of heart disease, preferably heart failure after acute myocardial infarction, and known to have a therapeutic effect.

In addition, the pharmaceutical composition according to the present disclosure may be used in heart disease, preferably with surgical treatment of heart failure after acute myocardial infarction.

Hereinafter, the present disclosure will be described in more detail through Examples. These Examples are to explain the present disclosure in more detail, and it will be apparent to those skilled in the art that the scope of the present disclosure is not limited to these Examples.

### <Example 1> Selection of Target Antigen Using Immunopeptidome

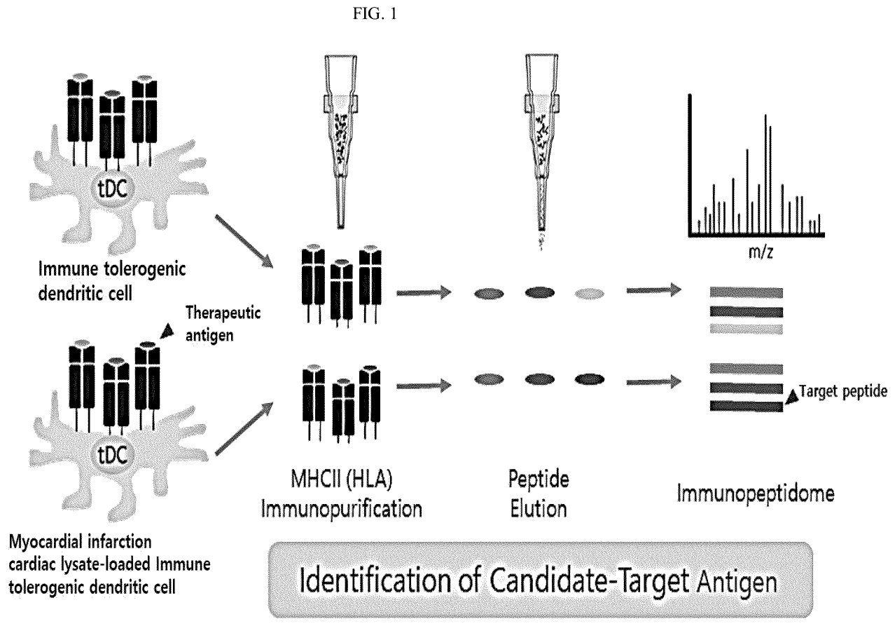

In order to select a therapeutic target antigen of the immune tolerogenic dendritic cells of the present disclosure, an immunopeptidome was used, and an experimental process was shown in FIG. 1. Specifically, mouse bone marrow-derived dendritic cells (BMDCs) were isolated and cultured for 4 hours in a medium containing ng/ml GM-CSF (JW Creagene), 2 ng/ml IL-4 (JW Creagene), and 10 ng/ml TNF-α (BD Pharmingen) to differentiate into immature dendritic cells (imDCs).

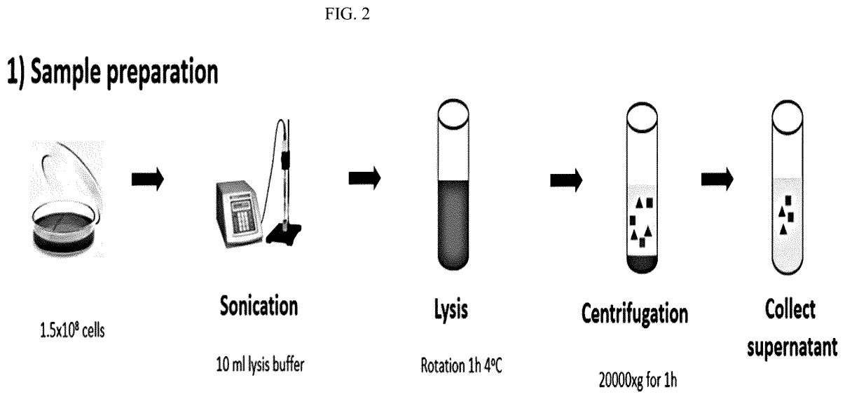

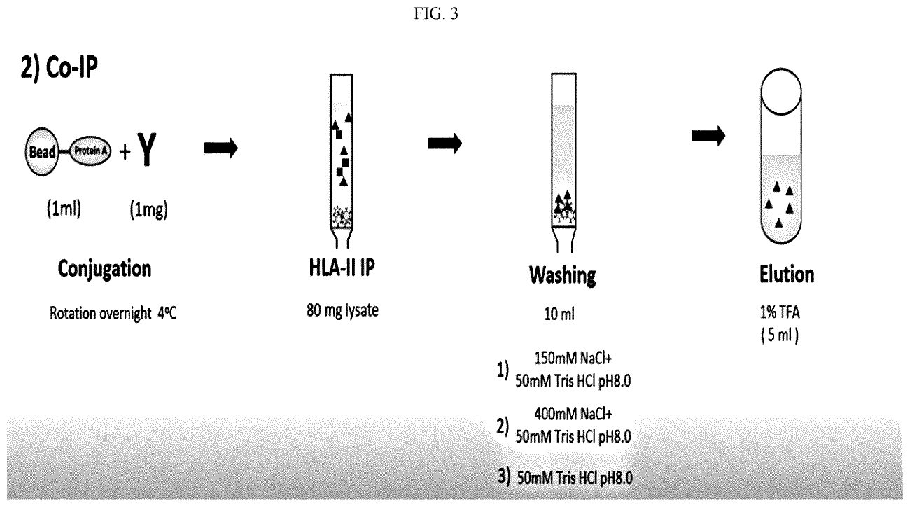

In addition, myocardial infarction was induced, a protein (heart lysate) of the myocardial infarction area of the mouse on day 1 was isolated, treated with the imDC, and cultured for 4 hours to differentiate into immune tolerogenic dendritic cells. A lysate obtained by sonicating the differentiated cells (1.5×10⁸) was centrifuged at 20000×g for 1 hour, and then a supernatant was obtained (FIG. 2). Thereafter, MHC II (HLA-II) of immune tolerogenic dendritic cells was conjugated to the beads with protein A at 4° C. overnight. Thereafter, the conjugated beads were treated with 80 mg of the supernatant of the same myocardial infarction heart lysate and immunopurification using HLA-II was performed. After the immunopurification, the beads were washed using 150 mM NaCl and 50 mM Tris HCl pH 8.0, 400 mM NaCl and 50 mM Tris HCl pH 8.0, and 50 mM Tris HCl pH 8.0 solutions, and the antigen was eluted with 5 ml of 1% TFA solution (FIG. 3). Thereafter, an immunopeptidome for target antigen discovery was performed using the eluted antigen.

As a result, Vimentin, Myl3, and Thymosin b4 were selected as target antigens for the treatment of heart failure after myocardial infarction in immune tolerogenic dendritic cells.

### <Example 2> Confirmation of Expression of Target Antigen in Myocardial Infarction Animal Model

<2-1> Confirmation of Expression of Target Antigen According to Induction of Myocardial Infarction

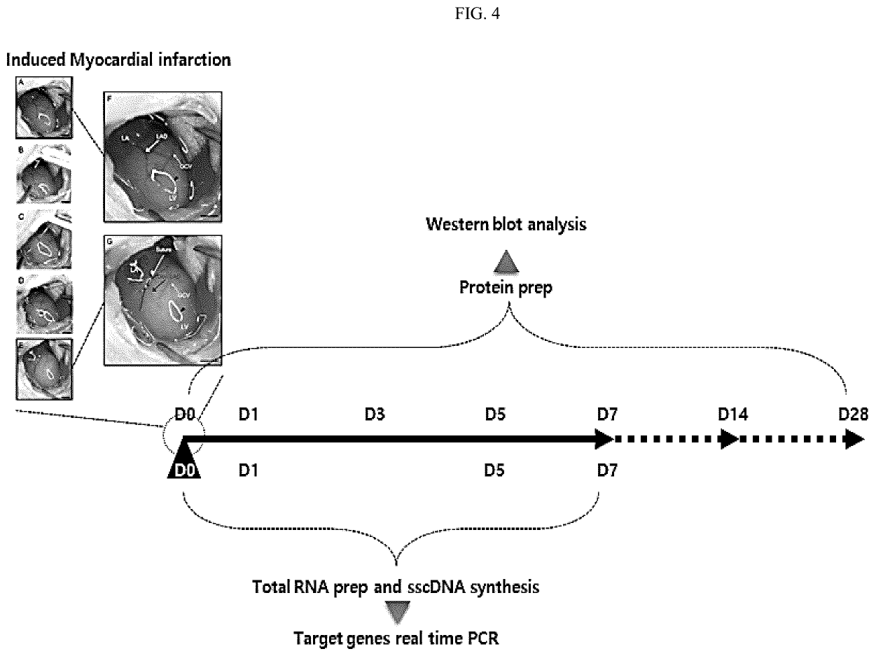

The expression of a target antigen for treatment of heart failure after myocardial infarction according to the present disclosure was confirmed in an animal model. Specifically, myocardial infarction was induced by suturing the left anterior descending branch (LAD) of the mouse coronary artery to induce coronary artery ligation. Then, on the day (D0), 1 day (D1), 5 days (D5), 7 days (D7), 14 days (D14), and 28 days (D28) of induction of myocardial infarction, the protein expression of a target antigen vimentin was analyzed by Western blot using an anti-vimentin antibody (abcam, ab92547) (FIG. 4). In addition, in order to confirm the expression of vimentin mRNA, RNA was isolated from heart tissue on the day (D0), 1 day (D1), 5 days (D5), and 7 days (D7) of induction of myocardial infarction and analyzed by real time PCR. Primers used in the real time PCR were as follows: mus_vimentin Fwd (AAT GCT TCT CTG GCA CGT CT) (SEQ ID NO: 1), mus_vimentin Rvs (GCT CCT GGA TCT CTT CAT CG) (SEQ ID NO: 2).

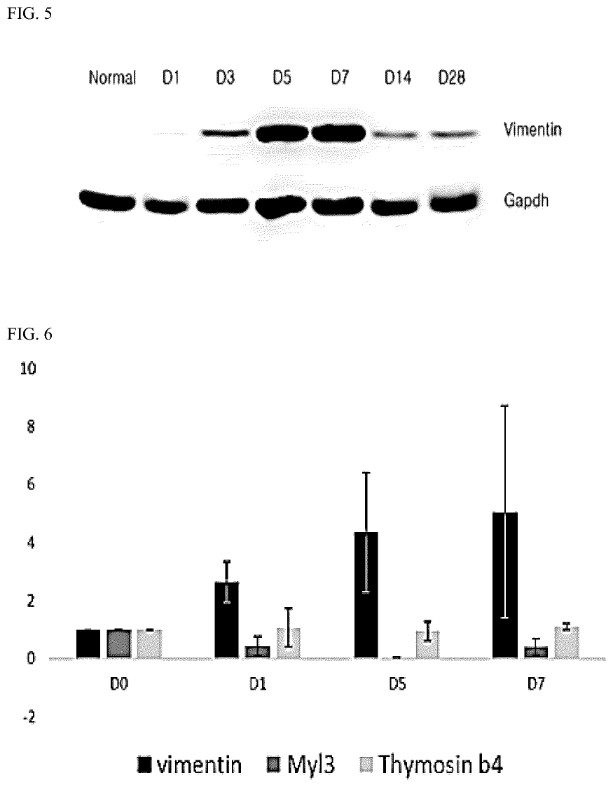

As a result, as illustrated in FIG. 5, it was confirmed that the expression of vimentin increased from day 1 after induction of myocardial infarction, and the expression of vimentin was highest on day 7 after induction of myocardial infarction. In addition, it was confirmed that in the same as the western blot result, the expression of vimentin mRNA also increased from day 1, and the expression of vimentin mRNA was the highest on day 7 after induction of myocardial infarction, and there was no significant difference in the expressions of Myl3 and Thymosin b4 (FIG. 6).

<2-2> Confirmation of Expression of Target Antigen in Myocardial Infarction Cells

The expression of the target antigen according to induction of myocardial infarction was confirmed in myocardial infarction cells of mice of Example 2-1. Specifically, among the mice in which myocardial infarction was induced in Example 2-1, D1, D3, D5, and D7 mice were humanely sacrificed, whole blood was obtained, and myocardial infarction sites of the hearts of D1, D3, and D5 mice were obtained. The obtained whole blood was centrifuged to isolate serum, and the amount of vimentin in the isolated serum was analyzed by ELISA.

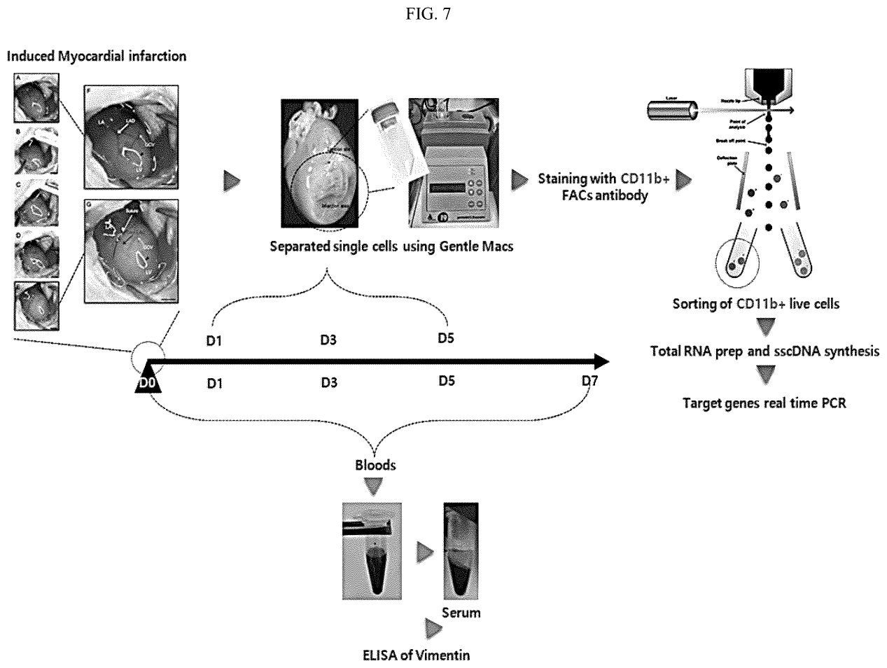

In addition, the obtained myocardial infarction site was isolated into single cells using Gentle Macs. The isolated cells were analyzed using Fluorescence-activated cell sorting (FACs) and analyzed after reacting with a CD11b+ FACs antibody. Thereafter, CD11b-positive cells were isolated, total RNA and sscDNA of the isolated cells were synthesized, and the expression of vimentin was confirmed by real time PCR (FIG. 7).

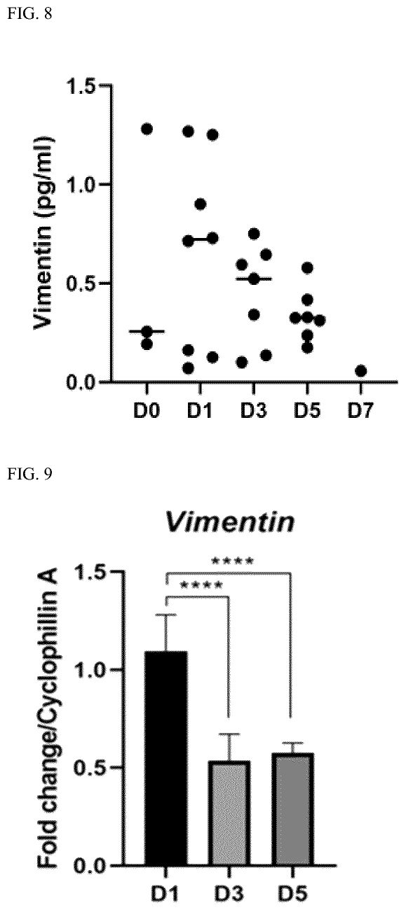

As a result, as illustrated in FIG. 8, it was confirmed that the content of vimentin in the obtained mouse serum was increased in D1 compared to D0, but the contents of vimentin decreased from D3 to D7.

In addition, it was confirmed that the expression of vimentin in CD11b+ macrophages of the myocardial infarction site was the highest at D1 and gradually decreased toward D5 (FIG. 9).

### <Example 3> Verification of Target Antigen of Immune Tolerogenic Dendritic Cells and Confirmation of Regulation of Immune-Related Factor Expression (In Vitro)

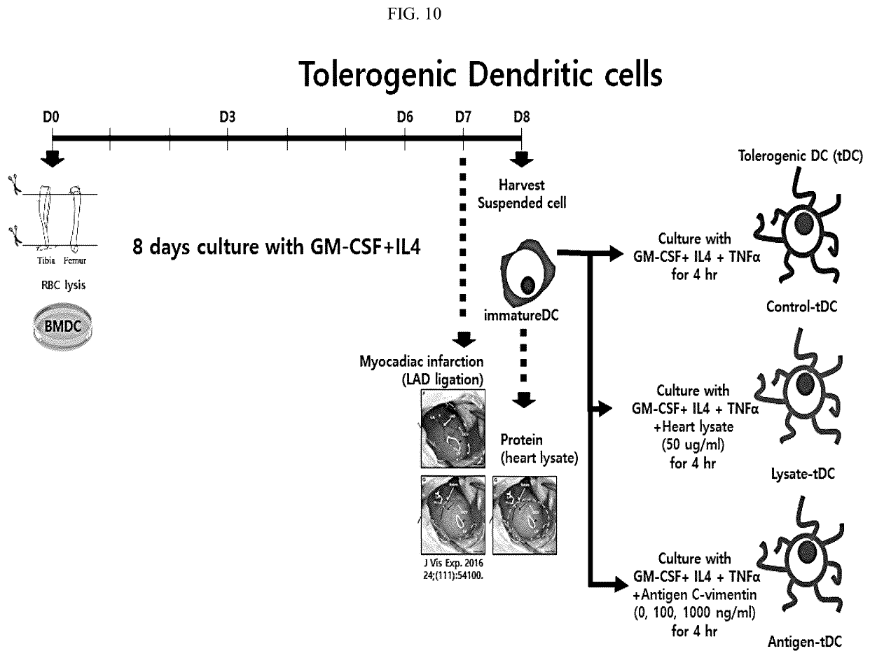

An in vitro model was used to verify a target antigen of the present disclosure. Specifically, bone marrow was obtained from the Tibia and Femur of mice, and red blood cells (RBCs) were lysed in the obtained bone marrow to obtain bone marrow dendritic cells (BMDCs). The obtained BMDCs were cultured for 8 days, and then immature dendritic cells were differentiated into immune tolerogenic DCs (tDCs). Specifically, three types of tDCs were prepared as follows: Control-tDC prepared by treating the bone marrow dendritic cell-derived dendritic cell precursor with GM-CSF+IL4 and TNF-α; Lysate-tDC differentiated by treating GM-CSF+IL4 and TNF-α and treating a heart lysate at 50 μg/ml; and Antigen-tDC differentiated by treating GM-CSF+IL4 and TNF-α and treating citrullinated vimentin (C-Vimentin) as an antigen at 10, 100, and 1000 ng/ml, respectively (FIG. 10). Then, the expression of factors related to immune regulation was confirmed by real time PCR.

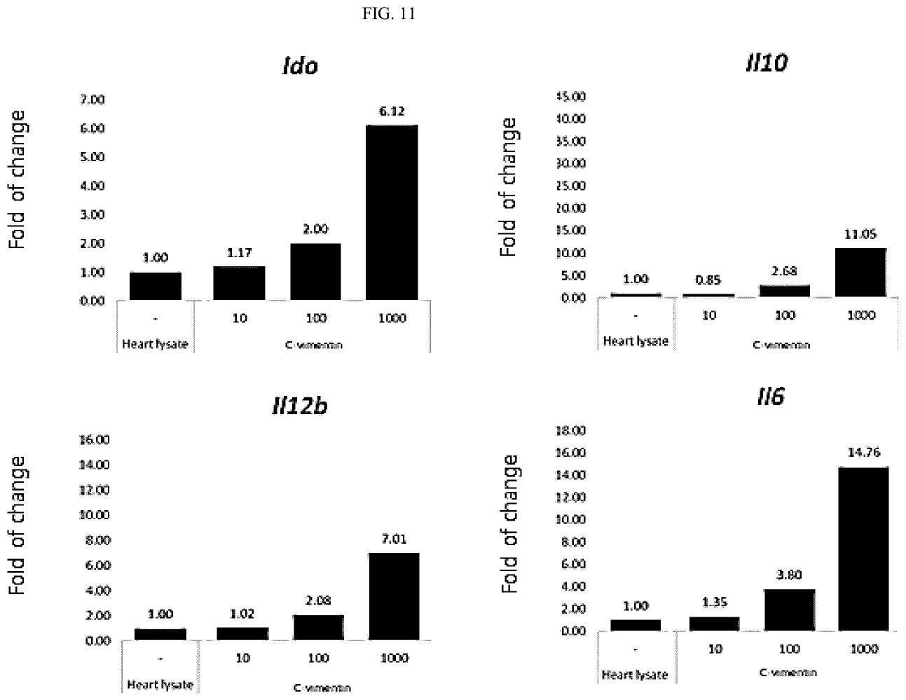

As a result, as illustrated in FIG. 11, it was confirmed that the expression of Ido, IL-10, IL-12b, and IL-6 was increased in a concentration-dependent manner in antigen-tDC treated with C-Vimentin.

In the case of treating C-Vimentin at an excessively high concentration, there is a possibility that hyperdifferentiation may be caused, and subsequent experiments were performed by setting the concentration of C-Vimentin to 100 ng/ml.

### <Example 4> Confirmation of Treatment Effect in Myocardial Infarction Animal Model (In Vivo)

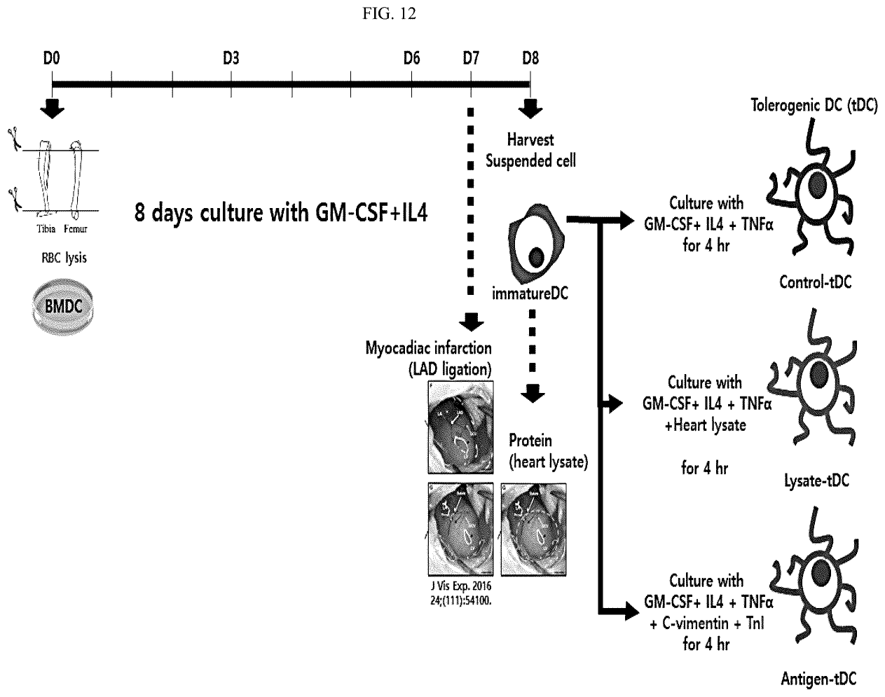

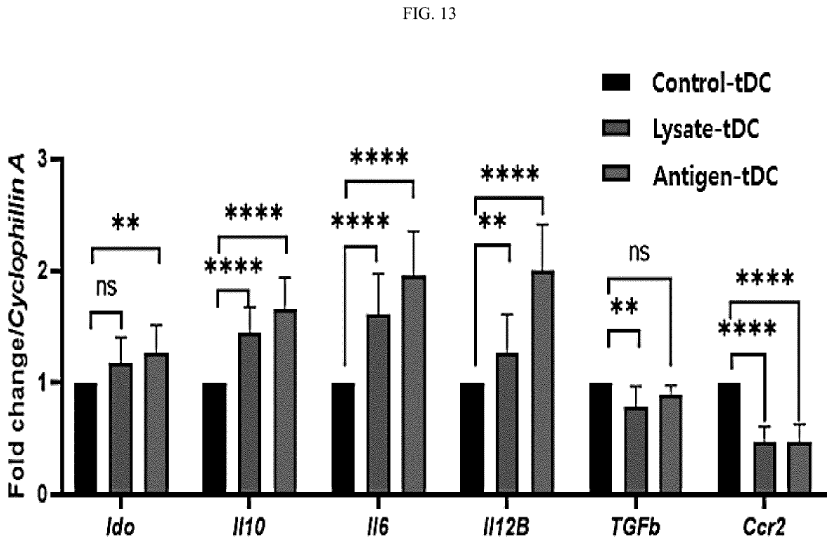

Bone marrow was obtained from the Tibia and Femur of mice, and red blood cells (RBCs) were lysed in the obtained bone marrow to obtain bone marrow dendritic cells (BMDCs). The obtained BMDCs were cultured for 8 days, and then immature dendritic cells were differentiated into immune tolerogenic DCs (tDCs). Specifically, three types of tDCs were prepared as follows: Control-tDC prepared by treating the bone marrow dendritic cell-derived dendritic cell precursor with GM-CSF+IL4 and TNF-α; Lysate-tDC differentiated by treating GM-CSF+IL4 and TNF-α and treating a heart lysate at 50 μg/ml; and Antigen-tDC differentiated for 4 hours by treating GM-CSF+IL4 and TNF-α and co-treating 100 ng/ml of citrullinated vimentin (C-Vimentin) as an antigen and 1 ng/ml of Troponin I (TnI) as a protein known as a representative antigen of myocardial infarction (FIG. 12). As a result of confirming the expression of factors related to immune regulation by real-time PCR, as illustrated in FIG. 13, the expression of Ido, IL-10, IL-12b, and IL-6 gradually increased significantly in the Antigen-tDC according to the present disclosure. In particular, it was confirmed that the expression of CCR2 was significantly lowered compared to Control-tDC.

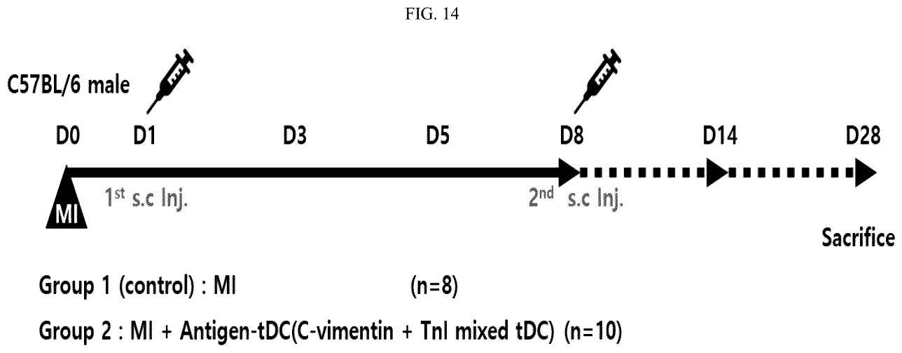

In addition, in order to confirm the effect in vivo, the animal model induced with myocardial infarction in the same manner as in Example <2-1> was divided into two Experimental Groups as follows: Experiment Group 1 (control): 8 C57BL/6 male mice induced with myocardial infarction, Experimental Group 2: 10 male mice injected with Antigen-tDC (1×10⁶ cells) according to the present disclosure after inducing myocardial infarction. In Experimental Group 2, Lysate-tDC or Antigen-tDC according to the present disclosure was administered on 1 day (D1) and 8 days (D8) after the induction of myocardial infarction, and euthanized on day 28 (D28) (FIG. 14).

In addition, the medial thickness (MT) was confirmed by staining the coronary arteries of each Experimental Group, and the infarct size and probability of survival were confirmed. After the mouse was sacrificed, the middle part of the heart was cut and made into a paraffin block, and tissue sections cut to a thickness of 5 μm were prepared. Masson trichome (MT) staining was performed to observe morphology and measure the degree of fibrosis of the myocardium. The infarct size was calculated as the percentage of the length of the ischemic area to the length of the central circumference of the left ventricular (LV) wall after MT staining of the heart tissue of the midline including the papillary muscle.

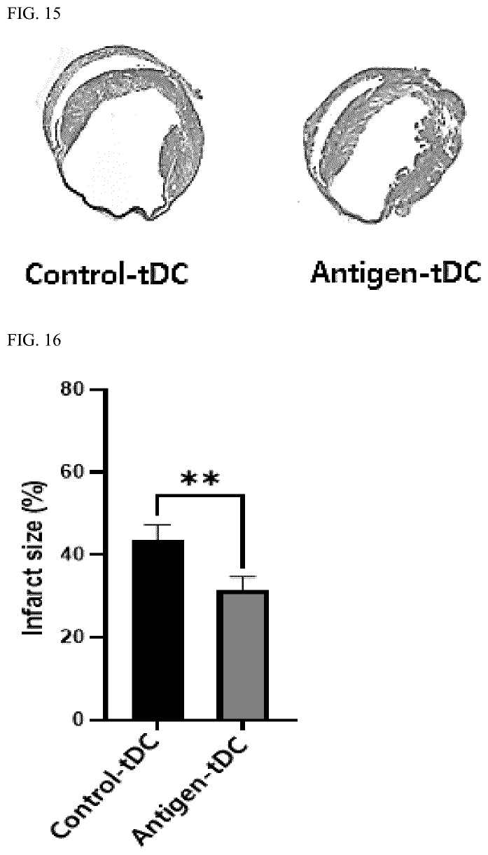

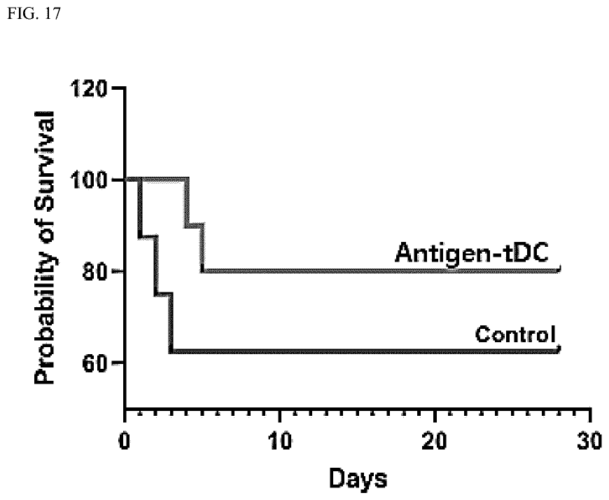

As a result, it was confirmed that since coronary MT in Experimental Group administered with Antigen-tDC according to the present disclosure was not significantly increased, excessive remodeling of the myocardium was suppressed (FIG. 15). In addition, it was confirmed that the infarct size was significantly reduced (FIG. 16), and the probability of survival was significantly increased (FIG. 17), so that the Antigen-tDC according to the present disclosure exhibited an excellent therapeutic effect on heart failure caused by myocardial infarction.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A preparation method of immune tolerogenic dendritic cells comprising culturing immature dendritic cells in a citrullinated vimentin-containing medium.

2. The preparation method of immune tolerogenic dendritic cells of claim 1, wherein the medium further comprises Troponin I.

3. The preparation method of the immune tolerogenic dendritic cells of claim 1, wherein the medium further comprises at least one selected from the group consisting of granulocyte macrophage colony stimulating factor (GM-CSF), interleukin-4 (IL-4) and tumor necrosis factor-α (TNF-α).

4. The preparation method of the immune tolerogenic dendritic cells of claim 1, wherein the culture is performed for 2 to 24 hours.

5. A method for inducing differentiation of immature dendritic cells into immune tolerogenic dendritic cells, comprising treating immature dendritic cells with citrullinated vimentin.

6. An immune tolerogenic dendritic cell comprising citrullinated vimentin as an antigen, prepared through the preparation method according to claim 1.

7. The immune tolerogenic dendritic cell of claim 6, wherein the immune tolerogenic dendritic cell further comprises Troponin I as an antigen.

8. The immune tolerogenic dendritic cell of claim 6, wherein the immune tolerogenic dendritic cell increases the expression of at least one factor selected from the group consisting of Ido, IL-10, IL-12b and IL-6.

9. The immune tolerogenic dendritic cell of claim 6, wherein the immune tolerogenic dendritic cell suppresses the expression of at least one factor selected from the group consisting of TGF-beta and CCR2.

10. A method for preventing or treating heart diseases comprising administering to a subject immune tolerogenic dendritic cells prepared by the preparation method according to claim 1.

11. The method for preventing or treating heart diseases of claim 10, wherein the heart disease is at least one selected from the group consisting of myocardial infarction, myocarditis, heart failure and cardiomyopathy.

12. The method for preventing or treating heart diseases of claim 11, wherein the heart failure is heart failure after acute myocardial infarction.


SEQUENCE LISTING (2 sequences)

SEQ ID NO: 1 - DNA, 20 bp, synthetic construct
  source 1..20: mol_type = other DNA
aatgcttctctggcacgtct

SEQ ID NO: 2 - DNA, 20 bp, synthetic construct
  source 1..20: mol_type = other DNA
gctcctggatctcttcatcg